(19) United States
(12) Patent Application Publication
(10) Pub. No.: US 2021/0147485 A1
(43) Pub. Date: May 20, 2021

(54) PEPTIDE LIGANDS FOR BINDING TO INTEGRIN

(71) Applicant: BicycleTx Limited, Cambridge (GB)

(72) Inventors: Daniel TEUFEL, Cambridge (GB); Gemma MUDD, Cambridge (GB); Silvia PAVAN, Cambridge (GB)

(21) Appl. No.: 17/254,464

(22) PCT Filed: Jun. 18, 2019
(86) PCT No.: PCT/EP2019/066010

§ 371 Date: Dec. 21, 2020

(30) Foreign Application Priority Data
Jun. 22, 2018 (GB) 1810329.1

(51) Int. Cl. C07K 7/08; A61K 47/64

(52) U.S. Cl. CPC C07K 7/08; A61K 47/64

(57) ABSTRACT

A peptide ligand specific for integrin αvβ3 comprising a polypeptide comprising three residues selected from cysteine, L-2,3-diaminopropionic acid (Dap), N-beta-alkyl-L-2,3-diaminopropionic acid (N-AlkDap) and N-beta-haloalkyl-L-2,3-diaminopropionic acid (N-HAlkDap), with the proviso that at least one of said three residues is selected from Dap, N-AlkDap or N-HAlkDap, the said three residues being separated by at least two loop sequences, and a molecular scaffold, the peptide being linked to the scaffold by covalent alkylamino linkages with the Dap or N-AlkDap or N-HAlkDap residues of the polypeptide and by thioether linkages with the cysteine residues of the polypeptide when the said three residues include cysteine, such that two polypeptide loops a formed on the molecular scaffold. Also provided are drug conjugates comprising the peptide ligands conjugated to one or more effector groups and pharmaceutical compositions comprising the conjugates.

### TECHNICAL FIELD

The present invention relates to peptide ligands which are high affinity binders of integrin αvβ3. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by integrin αvβ3. In particular, the invention relates to peptide ligands of this type having novel chemistries for forming two or more bonds between a peptide and a scaffold molecule.

### BACKGROUND OF THE INVENTION

Different research teams have previously tethered peptides to scaffold moieties by forming two or more thioether bonds between cysteine residues of the peptide and suitable functional groups of a scaffold molecule. For example, methods for the generation of candidate drug compounds by linking cysteine-containing peptides to a molecular scaffold as for example tris(bromomethyl) benzene are disclosed in WO 2004/077062 and WO 2006/078161.

The advantage of utilising cysteine thiols for generating covalent thioether linkages in order to achieve cyclisation resides is their selective and biorthogonal reactivity. Thiol-containing linear peptides may be cyclised with a thiol-reactive scaffold compound such as 1,3,5 tris-bromomethylbenzene (TBMB) to form Bicyclic Peptides, and the resultant product contains three thioethers at the benzylic locations. The overall structure of the linear peptide with TBMB to form a looped bicyclic peptide with thioether linkages is shown in FIG. 1.

A need exists for alternative chemistries for coupling peptides to scaffold moieties to form looped peptide structures employing suitable replacements of the thioether moiety, thereby achieving compatibility with different peptides, changes in physiochemical properties such as improved solubility, changes in biodistribution and other advantages.

WO2011/018227 describes a method for altering the conformation of a first peptide ligand or group of peptide ligands, each peptide ligand comprising at least two reactive groups separated by a loop sequence covalently linked to a molecular scaffold which forms covalent bonds with said reactive groups, to produce a second peptide ligand or group of peptide ligands, comprising assembling said second derivative or group of derivatives from the peptide(s) and scaffold of said first derivative or group of derivatives, incorporating one of: (a) altering at least one reactive group; or (b) altering the nature of the molecular scaffold; or (c) altering the bond between at least one reactive group and the molecular scaffold; or any combination of (a), (b) or (c).

Our earlier pending applications PCT/EP2017/083953 and PCT/EP2017/083954 filed 20 Dec. 2017 describe bicycle peptides in which one or more thioether linkages to the scaffold molecule have been replaced by alkylamino linkages.

Integrins are heterodimeric matrix receptors that anchor cells to substrates and transmit externally derived signals across the plasma membrane. Integrin αvβ3 is involved in the osteoclast-mediated bone resorption, both in vivo and in vitro. This heterodimer molecule recognizes the amino acid motif Arg-Gly-Asp (RGD) contained in bone matrix proteins such as osteopontin and bone sialoprotein. Integrin αvβ3 is expressed in an osteoclast and its expression is modulated by resorptive steroids and cytokines. Based on blocking experiments, αvβ3 integrin has been identified as a major functional adhesion receptor on osteoclasts. Inhibitors of integrin αvβ3 reduce the capacity of osteoclasts to bind to and resorb bone. Integrin αvβ3 plays a major role in the function of osteoclasts and inhibitors of this integrin are being considered for treating or preventing osteoporosis, osteolytic metastases, and malignancy-induced hypercalcemia.

There are many bone diseases that are related to osteolysis that is mediated by osteoclasts. Osteoporosis is the most common one that is induced when resorption and formation of bone are not coordinated and bone breakdown overrides bone building. Osteoporosis is also caused by other conditions, such as hormonal imbalance, diseases, or medications (e.g., corticosteroids or anti-epileptic agents). Bone is one of the most common sites of metastasis by human breast, prostate, lung and thyroid cancers, as well as other cancers. Osteoporosis may also result from post-menopausal estrogen deficiency. Secondary osteoporosis may be associated with rheumatoid arthritis. Bone metastasis shows a very unique step of osteoclastic bone resorption that is not seen in metastasis of other organs. It is widely accepted that osteolysis that is associated with cancer is essentially mediated by osteoclasts, which seem to be activated and may be indirectly activated through osteoblasts or directly by tumor products. In addition, hypercalcemia (increased blood-calcium concentration) is an important complication of osteolytic bone diseases. It occurs relatively frequently in patients with extensive bone destruction, and is particularly common in breast, lung, renal, ovarian and pancreatic carcinomas and in myeloma.

Disintegrins are a family of low-molecular-weight RGD-containing peptides that bind specifically to integrins αIIbβ3, α5β1 and αvβ3 expressed on platelets and other cells including vascular endothelial cells and some tumor cells. In addition to their potent antiplatelet activity, studies of disintegrins have revealed new uses in the diagnosis of cardiovascular diseases and the design of therapeutic agents in arterial thrombosis, osteoporosis and angiogenesis-related tumor growth and metastasis. Rhodostomin (Rho), a disintegrin derived from the venom of Colloselasma rhodostoma, has been found to inhibit platelet aggregation in vivo and in vitro through the blockade of platelet glycoprotein αIIbβ3.

The role of αvβ3 integrin in bone diseases has been well documented (Ross et al (2006) Journal of Clinical Investigation 116(5); Rodan et al (1997) Journal of Endocrinology 154, S47-S56; Teitelbaum (2005) Journal of Clinical Endocrinology and Metabolism 90(4), 2466-2468; Teitelbaum (2000) Journal of Bone and Mineral Metabolism 18, 344-349; Nakamura et al (2007) Journal of Bone and Mineral Metabolism 25, 337-344; Duong et al (1999) Journal of Bone and Mineral Metabolism 17, 1-6; and Teti et al (2002) Calcified Tissue International 71, 293-299). In addition to bone diseases, αvβ3 integrin plays an important role in angiogenesis and tumor growth in conditions not related to bone diseases.

Our earlier pending application GB1800598.3 filed on 15 Jan. 2018, describes bicycle peptide ligands having high binding affinity for integrin αvβ3. These applications further describe conjugates of the peptide ligands with therapeutic agents, in particular with cytotoxic agents.

### SUMMARY OF THE INVENTION

The present inventors have found that replacement of thioether linkages in looped peptides having affinity for integrin αvβ3 by alkylamino linkages results in looped peptide conjugates that display similar affinities to integrin αvβ3 as the corresponding conjugates made with all thioether linkages. The replacement of thioether linkages by alkylamino linkages is expected to result in improved solubility and/or improved oxidation stability of the conjugates according to the present invention.

Accordingly, in a first aspect the present invention provides a peptide ligand specific for integrin αvβ3 comprising a polypeptide comprising three residues selected from cysteine, L-2,3-diaminopropionic acid (Dap), N-beta-alkyl-L-2,3-diaminopropionic acid (N-AlkDap) and N-beta-haloalkyl-L-2,3-diaminopropionic acid (N-HAlkDap), with the proviso that at least one of said three residues is selected from Dap, N-AlkDap or N-HAlkDap, the said three residues being separated by at least two loop sequences, and a molecular scaffold, the peptide being linked to the scaffold by covalent alkylamino linkages with the Dap or N-AlkDap or N-HAlkDap residues of the polypeptide and by thioether linkages with the cysteine residues of the polypeptide when the said three residues include cysteine, such that two polypeptide loops are formed on the molecular scaffold.

Suitably, the peptide ligand comprises an amino acid sequence selected from:

Cᵢ-X₁-Cᵢᵢ-X₂-Cᵢᵢᵢ

wherein:
Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap; and
X₁ and X₂ represent the amino acid sequences between the Cysteine, Dap, N-AlkDap or N-HAlkDap residues, wherein each of X₁ and X₂ independently is a loop sequence of from 3 to 9 amino acid residues.

It can be seen that the derivatives of the invention comprise a peptide loop coupled to a scaffold by at least one alkylamino linkage to Dap or N-AlkDap of N-HAlkDap residues and up to two thioether linkages to cysteine.

The prefix “alkyl” in N-AlkDap and N-HAlkDap refers to an alkyl group having from one to four carbon atoms, preferably methyl. The prefix “halo” is used in this context in its normal sense to signify alkyl groups having one or more, suitably one, fluoro-, chloro-, bromo- or iodo-substituents.

When cysteine is present, the thioether linkage(s) provides an anchor during formation of the cyclic peptides as explained further below. In these embodiments, the thioether linkage is suitably a central linkage of the bicyclic peptide conjugate, i.e. in the peptide sequence two residues forming alkylamino linkages in the peptide are spaced from and located on either side of a cysteine residue forming the thioether linkage. In these preferred embodiments, the looped peptide structure is therefore a Bicycle peptide conjugate having a central thioether linkage and two peripheral alkylamino linkages. In alternative embodiments, the thioether linkage is placed at the N-terminus or C-terminus of the peptides, the central linkage and the other terminal linkage being selected from Dap, N-AlkDap or N-HAlkDap.

In embodiments of the invention all three of Cᵢ, Cᵢᵢ, and Cᵢᵢᵢ may be Dap or N-AlkDap or N-HAlkDap. In these embodiments, the peptide ligands of the invention are suitably Bicycle conjugates having a central alkylamino linkage and two peripheral alkylamino linkages, the peptide forming two loops sharing the central alkylamino linkage. In these and other embodiments, Cᵢ, Cᵢᵢ, and Cᵢᵢᵢ are suitably selected from N-AlkDap or N-HAlkDap, most suitably N-AlkDap, because of favourable reaction kinetics with the alkylated Daps.

Suitably, the peptide ligand of the invention is a high affinity binder of human, mouse and dog integrin αvβ3, in particular it is suitably a high affinity binder of human integrin αvβ3 Suitably the binding affinity K; is less than about 1000 nM, less than about 500 nM, less than about 100 nM, less than about 50 nM, or less than about 25 nM. The binding affinity in the context of this specification refers to the binding affinity as measured by the methods described below.

Suitably, the scaffold comprises a (hetero)aromatic or (hetero)alicyclic moiety.

In a further aspect, the present invention provides a drug conjugate comprising the peptide ligand according to the invention conjugated to one or more effector and/or functional groups such as a cytotoxic agent or a metal chelator. Suitably, the conjugate has the cytotoxic agent linked to the peptide ligand by a cleavable bond, such as a disulphide bond or a valine-citrulline linkage. Suitably, the cytotoxic agent is selected from DM1 or MMAE.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by integrin αvβ3.

### BRIEF DESCRIPTION OF THE DRAWINGS

FIG. 1 shows a schematic structure of a reference bicyclic peptide ligand exhibiting specific binding to integrin αvβ3;

FIG. 2 shows a schematic structure of a first bicyclic peptide ligand according to the present invention;

FIG. 3 shows a schematic structure of a second bicyclic peptide ligand according to the present invention;

FIG. 4 shows a schematic structure of a third bicyclic peptide ligand according to the present invention.

FIG. 5 shows a schematic structure of a fourth bicyclic peptide ligand according to the present invention;

FIG. 6 shows a schematic structure of a fifth bicyclic peptide ligand according to the present invention;

FIG. 7 shows a schematic structure of a sixth bicyclic peptide ligand according to the present invention;

FIG. 8 shows a schematic structure of a seventh bicyclic peptide ligand according to the present invention.

### DETAILED DESCRIPTION OF THE INVENTION

Unless defined otherwise, all technical and scientific terms used herein have the same meaning as commonly understood by those of ordinary skill in the art, such as in the arts of peptide chemistry, cell culture and phage display, nucleic acid chemistry and biochemistry.

Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

The present invention provides a looped peptide structure as defined in claim 1 comprising two peptide loops subtended between three linkages on the molecular scaffold, the central linkage being common to the two loops. The central linkage may be a thioether linkage formed to a cysteine residue of the peptide, or it is an alkylamino linkage formed to a Dap or N-AlkDap or N-HalkDap residue of the peptide. The two outer linkages are suitably alkylamino linkages formed to Dap or N-AlkDap or N-HalkDap residues of the peptide, or one of the outer linkages may be a thioether linkage formed to a cysteine residue of the peptide.

In one embodiment, the peptide ligands of the invention are fully cross-reactive with murine, dog, cynomolgus and human integrin αvβ3. In a yet further embodiment, the peptide ligands of the invention are selective for integrin αvβ3.

Suitably the binding affinity K for integrin αvβ3 is less than about 1000 nM, less than about 500 nM, less than about 250 nM, less than about 100 nM, or less than about 50 nM.

The amino acid sequences of various specific peptide ligands according to the present invention are defined in the accompanying claims.

When referring to amino acid residue positions within the bicycle peptide compounds of the invention, cysteine/Dap residues (Cᵢ, Cᵢᵢ, and Cᵢᵢᵢ) are omitted from the numbering as they are invariant, therefore, the numbering of amino acid residues within a representative bicycle compound is referred to as below:

-Cᵢ-L₁-D₂-H₃-M₄-E₅-Cᵢᵢ—R₆-G₇-D₈-M₉-D₁₀-Cᵢᵢᵢ—

Suitably, the peptides may be cyclised with TBMB (1,3,5-tris(bromomethyl)benzene) or 1,1′,1″-(1,3,5-triazinane-1,3,5-triyl)triprop-2-en-1-one (TATA) and yielding a tri-substituted structure. Cyclisation with TBMB and TATA occurs on Cᵢ, Cᵢᵢ, and Cᵢᵢᵢ.

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyn-group bearing amino acids that allow functionalisation with alkyn or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In an embodiment, the N-terminal modification comprises the addition of a molecular spacer group which facilitates the conjugation of effector groups and retention of potency of the bicyclic peptide to its target. The spacer group is suitably an oligopeptide group containing from about 5 to about 30 amino acids, such as an Ala, G-Sar10-A or bAla-Sar10-A group. In one embodiment, the spacer group is selected from bAla-Sar10-A.

For the purposes of this description, N- or C-terminal extensions to the bicycle core sequence are added to the left or right side of the sequence, separated by a hyphen. For example, an N-terminal βAla-Sar10-Ala tail would be denoted as:

βAla-Sar10-A-(SEQ ID NO: X)

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, C [symbol] disubstituted derivatives (for example, aminoisobutyric acid, Aib), and cyclo amino acids, a simple derivative being amino-cyclopropylcarboxylic acid.

In a further embodiment, the non-natural amino acid residues are selected from: 1-naphthylalanine; 2-naphthylalanine; cyclohexylglycine, phenylglycine; tert-butylglycine; 3,4-dichlorophenylalanine; cyclohexylalanine; and homophenylalanine.

In a yet further embodiment, the non-natural amino acid residues are selected from: 1-naphthylalanine; 2-naphthylalanine; and 3,4-dichlorophenylalanine. These substitutions enhance the affinity compared to the unmodified wildtype sequence.

In a yet further embodiment, the non-natural amino acid residues are selected from: 1-naphthylalanine. This substitution provided the greatest level of enhancement of affinity (greater than 7 fold) compared to wildtype.

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In all of the peptide sequences defined herein, one or more tyrosine residues may be replaced by phenylalanine. This has been found to improve the yield of the bicycle peptide product during base-catalyzed coupling of the peptide to the scaffold molecule.

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

The present invention includes all pharmaceutically acceptable (radio)isotope-labeled compounds of the invention wherein one or more atoms are replaced by atoms having the same atomic number, but an atomic mass or mass number different from the atomic mass or mass number usually found in nature, and compounds of the invention, wherein metal chelating groups are attached (termed “effector”) that are capable of holding relevant (radio)isotopes, and compounds of the invention, wherein certain functional groups are covalently replaced with relevant (radio)isotopes or isotopically labelled functional groups.

Examples of isotopes suitable for inclusion in the compounds of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled compounds of the invention, for example, those incorporating a radioactive isotope, are useful in drug and/or substrate tissue distribution studies, and to clinically assess the presence and/or absence of the integrin αvβ3 target on diseased tissues such as tumours and elsewhere. The compounds of the invention, can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Incorporation of isotopes into metal chelating effector groups, such as ⁶⁴Cu, ⁶⁷Ga, ⁶⁸Ga, and ¹⁷⁷Lu can be useful for visualizing tumour specific antigens employing PET or SPECT imaging.

Incorporation of isotopes into metal chelating effector groups, such as, but not limited to ⁹⁰Y, ¹⁷⁷Lu, and ²¹³Bi, can present the option of targeted radiotherapy, whereby metal-chelator-bearing compounds of the invention carry the therapeutic radionuclide towards the target protein and site of action.

Isotopically-labeled compounds of the invention can generally be prepared by conventional techniques known to those skilled in the art or by processes analogous to those described in the accompanying Examples using an appropriate isotopically-labeled reagent in place of the non-labeled reagent previously employed.

Specificity, in the context herein, refers to the ability of a ligand to bind or otherwise interact with its cognate target to the exclusion of entities which are similar to the target. For example, specificity can refer to the ability of a ligand to inhibit the interaction of a human enzyme, but not a homologous enzyme from a different species. Using the approach described herein, specificity can be modulated, that is increased or decreased, so as to make the ligands more or less able to interact with homologues or paralogues of the intended target. Specificity is not intended to be synonymous with activity, affinity or avidity, and the potency of the action of a ligand on its target (such as, for example, binding affinity or level of inhibition) are not necessarily related to its specificity.

Binding activity, as used herein, refers to quantitative binding measurements taken from binding assays, for example as described herein. Therefore, binding activity refers to the amount of peptide ligand which is bound at a given target concentration.

Multispecificity is the ability to bind to two or more targets. Typically, binding peptides are capable of binding to a single target, such as an epitope in the case of an antibody, due to their conformational properties. However, peptides can be developed which can bind to two or more targets; dual specific antibodies, for example, as known in the art as referred to above. In the present invention, the peptide ligands can be capable of binding to two or more targets and are therefore multispecific. Suitably, they bind to two targets, and are dual specific. The binding may be independent, which would mean that the binding sites for the targets on the peptide are not structurally hindered by the binding of one or other of the targets. In this case, both targets can be bound independently. More generally, it is expected that the binding of one target will at least partially impede the binding of the other.

There is a fundamental difference between a dual specific ligand and a ligand with specificity which encompasses two related targets. In the first case, the ligand is specific for both targets individually, and interacts with each in a specific manner. For example, a first loop in the ligand may bind to a first target, and a second loop to a second target. In the second case, the ligand is non-specific because it does not differentiate between the two targets, for example by interacting with an epitope of the targets which is common to both.

In the context of the present invention, it is possible that a ligand which has activity in respect of, for example, a target and an orthologue, could be a bispecific ligand. However, in one embodiment the ligand is not bispecific, but has a less precise specificity such that it binds both the target and one or more orthologues. In general, a ligand which has not been selected against both a target and its orthologue is less likely to be bispecific due to the absence of selective pressure towards bispecificity. The loop length in the bicyclic peptide may be decisive in providing a tailored binding surface such that good target and orthologue cross-reactivity can be obtained, while maintaining high selectivity towards less related homologues.

If the ligands are truly bispecific, in one embodiment at least one of the target specificities of the ligands will be common amongst the ligands selected, and the level of that specificity can be modulated by the methods disclosed herein. Second or further specificities need not be shared, and need not be the subject of the procedures set forth herein.

The peptide ligand compounds of the invention comprise, consist essentially of, or consist of, the peptide covalently bound to a molecular scaffold. The term “scaffold” or “molecular scaffold” herein refers to a chemical moiety that is bonded to the peptide at the alkylamino linkages and thioether linkage (when cysteine is present) in the compounds of the invention. The term “scaffold molecule” or “molecular scaffold molecule” herein refers to a molecule that is capable of being reacted with a peptide or peptide ligand to form the derivatives of the invention having alkylamino and, in certain embodiments, also thioether bonds. Thus, the scaffold molecule has the same structure as the scaffold moiety except that respective reactive groups (such as leaving groups) of the molecule are replaced by alkylamino and thioether bonds to the peptide in the scaffold moiety.

In embodiments, the scaffold is an aromatic molecular scaffold, i.e. a scaffold comprising a (hetero)aryl group. As used herein, “(hetero)aryl” is meant to include aromatic rings, for example, aromatic rings having from 4 to 12 members, such as phenyl rings. These aromatic rings can optionally contain one or more heteroatoms (e.g., one or more of N, O, S, and P), such as thienyl rings, pyridyl rings, and furanyl rings. The aromatic rings can be optionally substituted. “(hetero)aryl” is also meant to include aromatic rings to which are fused one or more other aryl rings or non-aryl rings. For example, naphthyl groups, indole groups, thienothienyl groups, dithienothienyl, and 5,6,7,8-tetrahydro-2-naphthyl groups (each of which can be optionally substituted) are aryl groups for the purposes of the present application. As indicated above, the aryl rings can be optionally substituted. Suitable substituents include alkyl groups (which can optionally be substituted), other aryl groups (which may themselves be substituted), heterocyclic rings (saturated or unsaturated), alkoxy groups (which is meant to include aryloxy groups (e.g., phenoxy groups)), hydroxy groups, aldehyde groups, nitro groups, amine groups (e.g., unsubstituted, or mono- or di-substituted with aryl or alkyl groups), carboxylic acid groups, carboxylic acid derivatives (e.g., carboxylic acid esters, amides, etc.), halogen atoms (e.g., Cl, Br, and I), and the like.

Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis.

In embodiments, the scaffold is a tris-methylene (hetero)aryl moiety, for example a 1,3,5-tris methylene benzene moiety. In these embodiments, the corresponding scaffold molecule suitably has a leaving group on the methylene carbons. The methylene group then forms the R₁ moiety of the alkylamino linkage as defined herein. In these methylene-substituted (hetero)aromatic compounds, the electrons of the aromatic ring can stabilize the transition state during nucleophilic substitution. Thus, for example, benzyl halides are 100-1000 times more reactive towards nucleophilic substitution than alkyl halides that are not connected to a (hetero)aromatic group.

In these embodiments, the scaffold and scaffold molecule have the general formula:

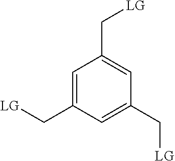

Where LG represents a leaving group as described further below for the scaffold molecule, or LG (including the adjacent methylene group forming the R₁ moiety of the alkylamino group) represents the alkylamino linkage to the peptide in the conjugates of the invention.

In embodiments, the group LG above may be a halogen such as, but not limited to, a bromine atom, in which case the scaffold molecule is 1,3,5-Tris(bromomethyl)benzene (TBMB). Another suitable molecular scaffold molecule is 2,4,6-tris(bromomethyl) mesitylene. It is similar to 1,3,5-tris(bromomethyl) benzene but contains additionally three methyl groups attached to the benzene ring. In the case of this scaffold, the additional methyl groups may form further contacts with the peptide and hence add additional structural constraint. Thus, a different diversity range is achieved than with 1,3,5-Tris(bromomethyl)benzene.

Another preferred molecule for forming the scaffold for reaction with the peptide by nucleophilic substitution is 1,3,5-tris(bromoacetamido)benzene (TBAB):

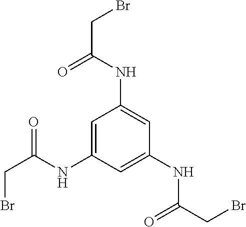 [SMILES: O=C(CBr)Nc1cc(NC(=O)CBr)cc(NC(=O)CBr)c1]

In other embodiments, the scaffold is a non-aromatic molecular scaffold, e.g. a scaffold comprising a (hetero)alicyclic group. As used herein, “(hetero)alicyclic” refers to a homocyclic or heterocyclic saturated ring. The ring can be unsubstituted, or it can be substituted with one or more substituents. The substituents can be saturated or unsaturated, aromatic or nonaromatic, and examples of suitable substituents include those recited above in the discussion relating to substituents on alkyl and aryl groups. Furthermore, two or more ring substituents can combine to form another ring, so that “ring”, as used herein, is meant to include fused ring systems. In these embodiments, the alicyclic scaffold is preferably 1,1′,1″-(1,3,5-triazinane-1,3,5-triyl)triprop-2-en-1-one (TATA).

In other embodiments the molecular scaffold may have a tetrahedral geometry such that reaction of four functional groups of the encoded peptide with the molecular scaffold generates not more than two product isomers. Other geometries are also possible; indeed, an almost infinite number of scaffold geometries is possible, leading to greater possibilities for peptide ligand diversification.

The peptides used to form the ligands of the invention comprise Dap or N-AlkDap or N-HAlkDap residues for forming alkylamino linkages to the scaffold. The structure of diaminopropionic acid is analogous to and isosteric that of cysteine that has been used to form thioether bonds to the scaffold in the prior art, with replacement of the terminal —SH group of cysteine by —NH₂:

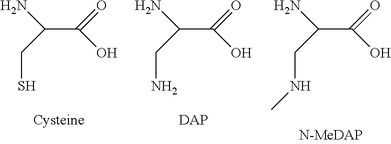

The term “alkylamino” is used herein in its normal chemical sense to denote a linkage consisting of NH or N(R₃) bonded to two carbon atoms, wherein the carbon atoms are independently selected from alkyl, alkylene, or aryl carbon atoms and R₃ is an alkyl group. Suitably, the alkylamino linkages of the invention comprise an NH moiety bonded to two saturated carbon atoms, most suitably methylene (—CH₂—) carbon atoms. The alkylamino linkages of the invention have general formula:

S—R₁—N(R₃)—R₂—P

### Wherein:

S represents the scaffold core, e.g. a (hetero)aromatic or (hetero)alicyclic ring as explained further below;
R₁ is C1 to C3 alkylene groups, suitably methylene or ethylene groups, and most suitably methylene (CH₂);
R₂ is the methylene group of the Dap or N-AlkDap side chain
R₃ is H or C1-4 alkyl including branched alkyl and cycloalkyl, for example methyl, wherein any of the alkyl groups is optionally halogenated; and
P represents the peptide backbone, i.e. the R₂ moiety of the above linkage is linked to the carbon atom in the peptide backbone adjacent to a carboxylic carbon of the Dap or N-AlkDap or N-HAlkDap residue.

Certain bicyclic peptide ligands of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes; and
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent.

It will be appreciated that salt forms are within the scope of this invention, and references to peptide ligands of the present invention include the salt forms of said compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydroiodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the present invention contain an amine function, these may form quaternary ammonium salts, for example by reaction with an alkylating agent according to methods well known to the skilled person. Such quaternary ammonium compounds are within the scope of the invention.

Several conjugated peptides may be incorporated together into the same molecule according to the present invention. For example two such peptide conjugates of the same specificity can be linked together via the molecular scaffold, increasing the avidity of the derivative for its targets. Alternatively, in another embodiment a plurality of peptide conjugates are combined to form a multimer. For example, two different peptide conjugates are combined to create a multispecific molecule. Alternatively, three or more peptide conjugates, which may be the same or different, can be combined to form multispecific derivatives. In one embodiment multivalent complexes may be constructed by linking together the molecular scaffolds, which may be the same or different.

The peptide ligands of the present invention may be made by a method comprising: providing a suitable peptide and a scaffold molecule; and forming the thioether (when cysteine is present) and alkylamino linkages between the peptide and the scaffold molecule.

The peptides for preparation of the peptide ligands of the invention can be made using conventional solid-phase synthesis from amino acid starting materials, which may include appropriate protecting groups as described herein. These methods for making peptides are well known in the art.

Suitably, the peptide has protecting groups on nucleophilic groups other than the —SH and amine groups intended for forming the alkylamino linkages. The nucleophilicity of amino acid side chains has been subject to several studies, and listed in descending order: thiolate in cysteines, amines in Lysine, secondary amine in Histidine and Tryptophan, guanidino amines in Arginine, hydroxyls in Serine/Threonine, and finally carboxylates in aspartate and glutamate. Accordingly, in some cases it may be necessary to apply protecting groups to the more nucleophilic groups on the peptide to prevent undesired side reactions with these groups.

In embodiments, the method comprises: synthesising a peptide having protecting groups on nucleophilic groups other than the amine groups intended for forming the alkylamino linkages and second protecting groups on the amine groups intended for forming alkylamino linkages, wherein the protecting groups on the amine groups intended for forming alkylamino linkages can be removed under conditions different than for the protecting groups on the other nucleophilic groups, followed by treating the peptide under conditions selected to deprotect the amine groups intended for forming alkylamino linkages without deprotecting the other nucleophilic groups. The coupling reaction to the scaffold is then performed, followed by removal of the remaining protecting groups to yield the peptide conjugate.

Suitably, the method comprises reacting, in a nucleophilic substitution reaction, the peptide having the reactive side chain —SH and amine groups, with a scaffold molecule having three or more leaving groups.

The term “leaving group” herein is used in its normal chemical sense to mean a moiety capable of nucleophilic displacement by an amine group. Any such leaving group can be used here provided it is readily removed by nucleophilic displacement by amine. Suitable leaving groups are conjugate bases of acids having a pKa of less than about 5. Non-limiting examples of leaving groups useful in the invention include halo, such as bromo, chloro, iodo, O-tosylate (OTos), O-mesylate (OMes), O-triflate (OTf) or O-trimethylsilyl (OTMS).

The nucleophilic substitution reactions may be performed in the presence of a base, for example where the leaving group is a conventional anionic leaving group. The present inventors have found that the yields of cyclised peptide ligands can be greatly increased by suitable choice of solvent and base (and pH) for the nucleophilic substitution reaction, and furthermore that the preferred solvent and base are different from the prior art solvent and base combinations that involve only the formation of thioether linkages. In particular, the present inventors have found that improved yields are achieved when using a trialkylamine base, i.e. a base of formula NR₁R₂R₃, wherein R₁, R₂ and R₃ are independently C1-C5 alkyl groups, suitably C2-C4 alkyl groups, in particular C2-C3 alkyl groups. Especially suitable bases are triethylamine and diisopropylethylamine (DIPEA). These bases have the property of being only weakly nucleophilic, and it is thought that this property accounts for the fewer side reactions and higher yields observed with these bases. The present inventors have further found that the preferred solvents for the nucleophilic substitution reaction are polar and protic solvents, in particular MeCN/H₂O containing MeCN and H₂O in volumetric ratios from 1:10 to 10:1, suitably from 2:10 to 10:2 and more suitably from 3:10 to 10:3, in particular from 4:10 to 10:4.

Additional binding or functional activities may be attached to the N or C terminus of the peptide covalently linked to a molecular scaffold. The functional group is, for example, selected from the group consisting of: a group capable of binding to a molecule which extends the half-life of the peptide ligand in vivo, and a molecule which extends the half-life of the peptide ligand in vivo. Such a molecule can be, for instance, HSA or a cell matrix protein, and the group capable of binding to a molecule which extends the half-life of the peptide ligand in vivo is an antibody or antibody fragment specific for HSA or a cell matrix protein. Such a molecule may also be a conjugate with high molecular weight PEGs.

In one embodiment, the functional group is a binding molecule, selected from the group consisting of a second peptide ligand comprising a peptide covalently linked to a molecular scaffold, and an antibody or antibody fragment. 2, 3, 4, 5 or more peptide ligands may be joined together. The specificities of any two or more of these derivatives may be the same or different; if they are the same, a multivalent binding structure will be formed, which has increased avidity for the target compared to univalent binding molecules. The molecular scaffolds, moreover, may be the same or different, and may subtend the same or different numbers of loops.

The functional group can moreover be an effector group, for example an antibody Fc region.

Attachments to the N or C terminus may be made prior to binding of the peptide to a molecular scaffold, or afterwards. Thus, the peptide may be produced (synthetically, or by biologically derived expression systems) with an N or C terminal peptide group already in place. Preferably, however, the addition to the N or C terminus takes place after the peptide has been combined with the molecular backbone to form a conjugate. For example, Fluorenylmethyloxycarbonyl chloride can be used to introduce the Fmoc protective group at the N-terminus of the peptide. Fmoc binds to serum albumins including HSA with high affinity, and Fmoc-Trp or Fmoc-Lys bind with an increased affinity. The peptide can be synthesised with the Fmoc protecting group left on, and then coupled with the scaffold through the alkylaminos. An alternative is the palmitoyl moiety which also binds HSA and has, for example been used in Liraglutide to extend the half-life of this GLP-1 analogue.

Alternatively, a conjugate of the peptide with the scaffold can be made, and then modified at the N-terminus, for example with the amine- and sulfhydryl-reactive linker N-e-maleimidocaproyloxy) succinimide ester (EMCS). Via this linker the peptide conjugate can be linked to other peptides, for example an antibody Fc fragment.

The binding function may be another peptide bound to a molecular scaffold, creating a multimer, another binding protein, including an antibody or antibody fragment; or any other desired entity, including serum albumin or an effector group, such as an antibody Fc region.

Additional binding or functional activities can moreover be bound directly to the molecular scaffold.

In embodiments, the scaffold may further comprise a reactive group to which the additional activities can be bound. Preferably, this group is orthogonal with respect to the other reactive groups on the molecular scaffold, to avoid interaction with the peptide. In one embodiment, the reactive group may be protected, and deprotected when necessary to conjugate the additional activities.

Accordingly, in a further aspect of the invention, there is provided a drug conjugate comprising a peptide ligand as defined herein conjugated to one or more effector and/or functional groups.

Effector and/or functional groups can be attached, for example, to the N or C termini of the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p 821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p 10444), the 18 amino acid ‘model amphipathic peptide’ (Oehlke et al (1998) Biochim Biophys Acts Volume 1414 p 127) and arginine rich regions of the HIV TAT protein. Non peptidic approaches include the use of small molecule mimics or SMOCs that can be easily attached to biomolecules (Okuyama et al (2007) Nature Methods Volume 4 p 153). Other chemical strategies to add guanidinium groups to molecules also enhance cell penetration (Elson-Scwab et al (2007) J Biol Chem Volume 282 p 13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half life in the range 12 to 60 hours. In a further embodiment, it will have a tβ half-life of a day or more. In a further embodiment still, it will be in the range 12 to 26 hours.

In one particular embodiment of the invention, the functional group conjugated to the looped peptide is selected from a metal chelator, which is suitable for complexing metal radioisotopes of medicinal relevance. Such effectors, when complexed with said radioisotopes, can present useful agents for cancer therapy. Suitable examples include DOTA, NOTA, EDTA, DTPA, HEHA, SarAr and others (Targeted Radionuclide therapy, Tod Speer, Wolters/Kuver Lippincott Williams & Wilkins, 2011).

Possible effector groups also include enzymes, for instance such as carboxypeptidase G2 for use in enzyme/prodrug therapy, where the peptide ligand replaces antibodies in ADEPT. In one particular embodiment of this aspect of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin and others.

In one further particular embodiment of the invention according to this aspect, the cytotoxic agent is selected from DM1 or MMAE.

DM1 is a cytotoxic agent which is a thiol-containing derivative of maytansine and has the following structure:

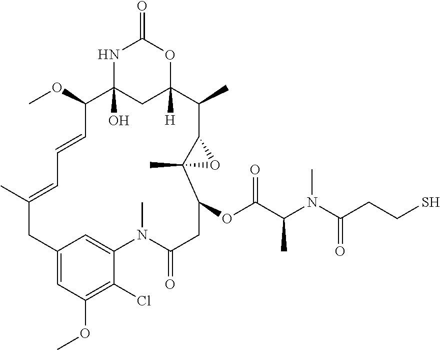

Monomethyl auristatin E (MMAE) is a synthetic antineoplastic agent and has the following structure:

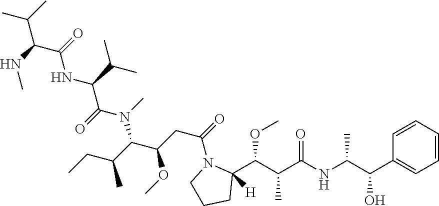 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In one embodiment, the cytotoxic agent is linked to the bicyclic peptide by a cleavable bond, such as a disulphide bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

Thus, in one embodiment, the cytotoxic agent is selected from a compound of formula:

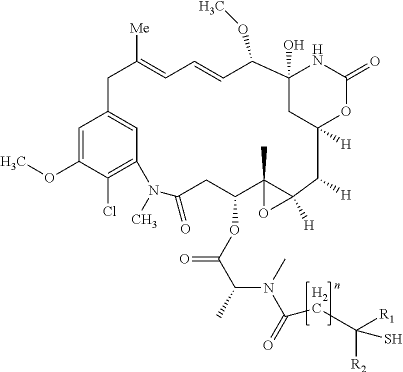

wherein n represents an integer selected from 1 to 10; and

R₁ and R₂ independently represent hydrogen or methyl groups.

In one embodiment of the compound of the above formula, n represents 1 and R₁ and R₂ both represent hydrogen (i.e. the maytansine derivative DM1).

In an alternative embodiment of the compound of the above formula, n represents 2, R₁ represents hydrogen and R₂ represents a methyl group (i.e. the maytansine derivative DM3).

In one embodiment of the compound, n represents 2 and R₁ and R₂ both represent methyl groups (i.e. the maytansine derivative DM4).

It will be appreciated that the cytotoxic agent can form a disulphide bond, and in a conjugate structure with a bicyclic peptide, the disulphide connectivity between the thiol-toxin and thiol-bicycle peptide is introduced through several possible synthetic schemes.

In one embodiment, the bicyclic peptide component of the conjugate has the following structure:

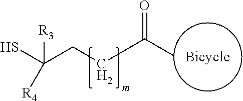

wherein m represents an integer selected from 0 to 10,
Bicycle represents any suitable looped peptide structure as described herein; and
R₃ and R₄ independently represent hydrogen or methyl.

Compounds of the above formula where R₃ and R₄ are both hydrogen are considered unhindered and compounds of the above formula where one or all of R₃ and R₄ represent methyl are considered hindered.

It will be appreciated that the bicyclic peptide of the above formula can form a disulphide bond, and in a conjugate structure with a cytotoxic agent described above, the disulphide connectivity between the thiol-toxin and thiol-bicycle peptide is introduced through several possible synthetic schemes.

In one embodiment, the cytotoxic agent is linked to the bicyclic peptide by the following linker:

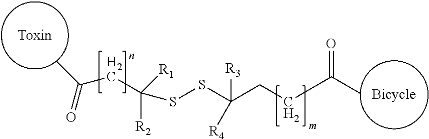

wherein R₁, R₂, R₃ and R₄ represent hydrogen or C1-C6 alkyl groups;
Toxin refers to any suitable cytotoxic agent defined herein;
Bicycle represents any suitable looped peptide structure as described herein;
n represents an integer selected from 1 to 10; and
m represents an integer selected from 0 to 10.

When R₁, R₂, R₃ and R₄ are each hydrogen, the disulphide bond is least hindered and most susceptible to reduction. When R₁, R₂, R₃ and R₄ are each alkyl, the disulphide bond is most hindered and least susceptible to reduction. Partial substitutions of hydrogen and alkyl yield a gradual increase in resistance to reduction, and concomitant cleavage and release of toxin. Preferred embodiments include: R₁, R₂, R₃ and R₄ all H; R₁, R₂, R₃ all H and R₄=methyl; R₁, R₂=methyl and R₃, R₄═H; R₁, R₃=methyl and R₂, R₄═H; and R₁, R₂═H, R₃, R₄═C1-C6 alkyl.

In one embodiment, the toxin of compound is a maytansine and the conjugate comprises a compound of the following formula:

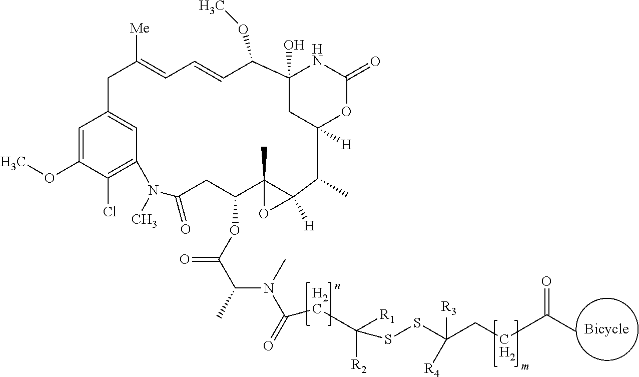

wherein R₁, R₂, R₃ and R₄ are as defined above;
Bicycle represents any suitable looped peptide structure as defined herein;
n represents an integer selected from 1 to 10; and
m represents an integer selected from 0 to 10.

Further details and methods of preparing the above-described conjugates of bicycle peptide ligands with toxins are described in detail in our published patent applications WO2016/067035 and WO2017/191460. The entire disclosure of these applications is expressly incorporated herein by reference.

The linker between the toxin and the bicycle peptide may comprise a triazole group formed by click-reaction between an azide-functionalized toxin and an alkyne-functionalized bicycle peptide structure (or vice-versa). In other embodiments, the bicycle peptide may contain an amide linkage formed by reaction between a carboxylate-functionalized toxin and the N-terminal amino group of the bicycle peptide.

The linker between the toxin and the bicycle peptide may comprise a cathepsin-cleavable group to provide selective release of the toxin within the target cells. A suitable cathepsin-cleavable group is valine-citrulline.

The linker between the toxin and the bicycle peptide may comprise one or more spacer groups to provide the desired functionality, e.g. binding affinity or cathepsin cleavability, to the conjugate. A suitable spacer group is para-amino benzyl carbamate (PABC) which may be located intermediate the valine-citrulline group and the toxin moiety.

Thus, in embodiments, the bicycle peptide-drug conjugate may have the following structure made up of Toxin-PABC-cit-val-triazole-Bicycle:

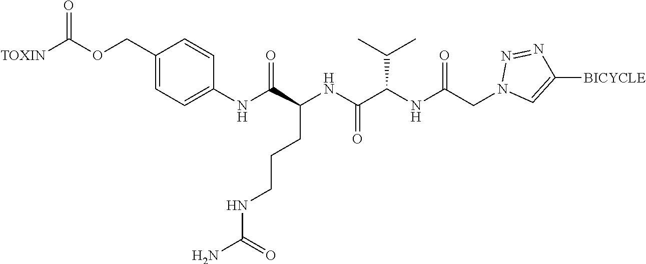

In further embodiments, the bicycle peptide-drug conjugate may have the following structure made up of Toxin-PABC-cit-val-dicarboxylate-Bicycle:

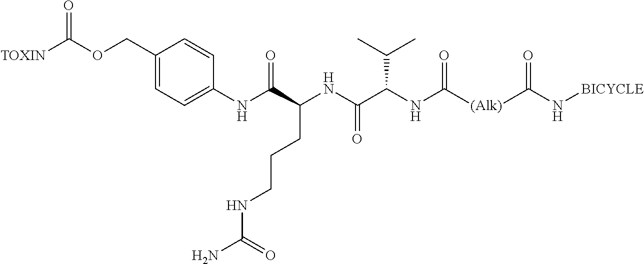

wherein (alk) is an alkylene group of formula CₙH₂ₙ wherein n is from 1 to 10 and may be linear or branched, suitably (alk) is n-propylene or n-butylene.

A detailed description of methods for the preparation of peptide ligand-drug conjugates according to the present invention is given in our earlier applications WO2016/067035 and PCT/EP2017/083954 filed 20 Dec. 2017, the entire contents of which are incorporated herein by reference.

Peptide ligands according to the present invention may be employed in in vivo therapeutic and prophylactic applications, in vitro and in vivo diagnostic applications, in vitro assay and reagent applications, and the like.

In general, the use of a peptide ligand can replace that of an antibody. Derivatives selected according to the invention are of use diagnostically in Western analysis and in situ protein detection by standard immunohistochemical procedures; for use in these applications, the derivatives of a selected repertoire may be labelled in accordance with techniques known in the art. In addition, such peptide ligands may be used preparatively in affinity chromatography procedures, when complexed to a chromatographic support, such as a resin. All such techniques are well known to one of skill in the art. Peptide ligands according to the present invention possess binding capabilities similar to those of antibodies, and may replace antibodies in such assays.

Diagnostic uses include any uses which to which antibodies are normally put, including test-strip assays, laboratory assays and immunodiagnostic assays.

Therapeutic and prophylactic uses of peptide ligands prepared according to the invention involve the administration of derivatives selected according to the invention to a recipient mammal, such as a human. Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected peptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

Generally, the present peptide ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a peptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected peptides according to the present invention having different specificities, such as peptides selected using different target derivatives, whether or not they are pooled prior to administration.

The route of administration of pharmaceutical compositions according to the invention may be any of those commonly known to those of ordinary skill in the art. For therapy, including without limitation immunotherapy, the selected antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counter-indications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by integrin αvβ3.

According to a further aspect of the invention, there is provided a method of preventing, suppressing or treating a disease or disorder mediated by integrin αvβ3, which comprises administering to a patient in need thereof an effector group and drug conjugate of the peptide ligand as defined herein.

In one embodiment, the integrin αvβ3 is mammalian integrin αvβ3. In a further embodiment, the mammalian integrin αvβ3 is human integrin αvβ3.

In one embodiment, the disease or disorder mediated by integrin αvβ3 is selected from bone disease (such as osteoporosis), cancer, and diseases involving angiogenesis.

In a further embodiment, the disease or disorder mediated by integrin αvβ3 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from cancer of the breast, lung, kidney, ovary and pancreas and myeloma.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described with reference to the following examples.

### EXAMPLES

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. All amino acids, unless noted otherwise, were used in the L-configurations. Peptides were purified using HPLC and following isolation they were modified with 1,3,5-tris(bromomethyl)benzene (TBMB, Sigma). For this, linear peptide was diluted with H₂O up to ˜35 mL, ˜500 μL of 100 mM TBMB in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Following lyophilisation, the modified peptide was purified as above, while replacing the Luna C8 with a Gemini C18 column (Phenomenex), and changing the acid to 0.1% trifluoroacetic acid. Pure fractions containing the correct TMB-modified material were pooled, lyophilised and kept at −20° C. for storage.

Alternatively, peptides were purified using HPLC and following isolation they were modified with 1,3,5-Triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

The following non-natural amino acid precursors were used for the preparation of the DAP and N-MeDAP modified peptides:

| Compound | CAS | Mw | Supplier |
|---|---|---|---|
| Fmoc-L-Dap(Boc,Me)-OH | 446847-80-9 | 440.49 | Iris Biotech GMBH |
| Fmoc-Dap(Boc)-OH | 162558-25-0 | 426.46 | Sigma Aldrich |

Integrin αvβ3 Competition Binding Assay

Affinity of the peptides of the invention for integrin αvβ3 (Ki) was determined using a competition fluorescence polarisation assay analogous to that described in Wang et al (2005) Bioconjug Chem 16(3), 729-34 using 5 nM peptide with the sequence: FITC-LC-GRGDSP as the ligand.

The peptide ligands of the examples and the reference examples were tested in the above mentioned assay.

### Reference Example 1

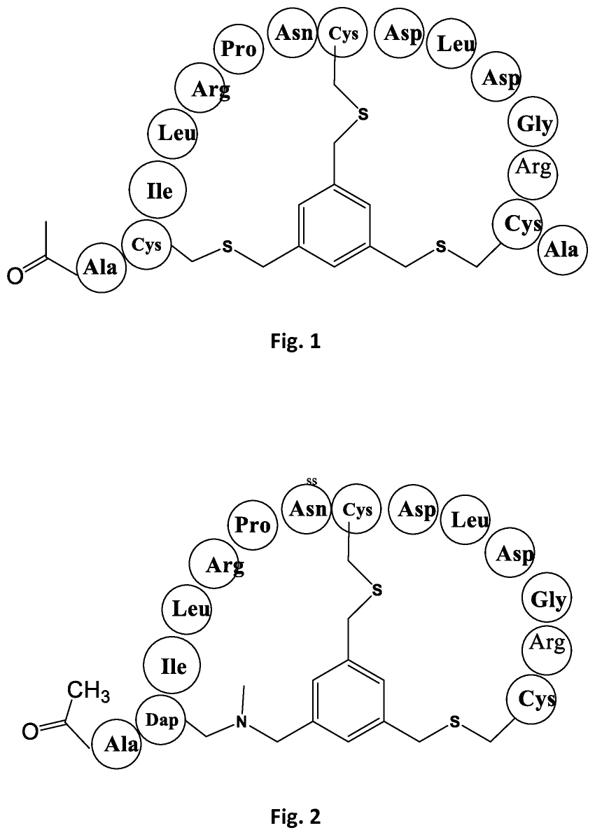

A first reference Bicyclic Peptide chosen for comparison of thioether to alkylamino scaffold linkage was designated BCY00009174. It is a bicycle conjugate of a thioether-forming peptide comprising three cysteine residues with a trimethylene benzene scaffold. The structure of this bicycle derivative is shown schematically in FIG. 1. The linear peptide before conjugation has sequence:

[Ac]ACILRPNCDLDGRCA

Conjugation to 1,3,5-tris(bromomethyl)benzene (TBMB, Sigma) was carried out as follows. The linear peptide was diluted with H₂O up to ˜35 mL, ˜500 μL of 100 mM TBMB in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Following lyophilisation, the modified peptide was purified with a Gemini C18 column (Phenomenex), and changing the acid to 0.1% trifluoroacetic acid. Pure fractions containing the correct TMB-modified material were pooled, lyophilised and kept at −20° C. for storage.

The resulting Bicycle derivative designated BCY00009174 showed high affinity to integrin αvβ3. The measured affinity (Ki) to integrin αvβ3 of the derivative was 10.4 nM.

### Examples 1-7

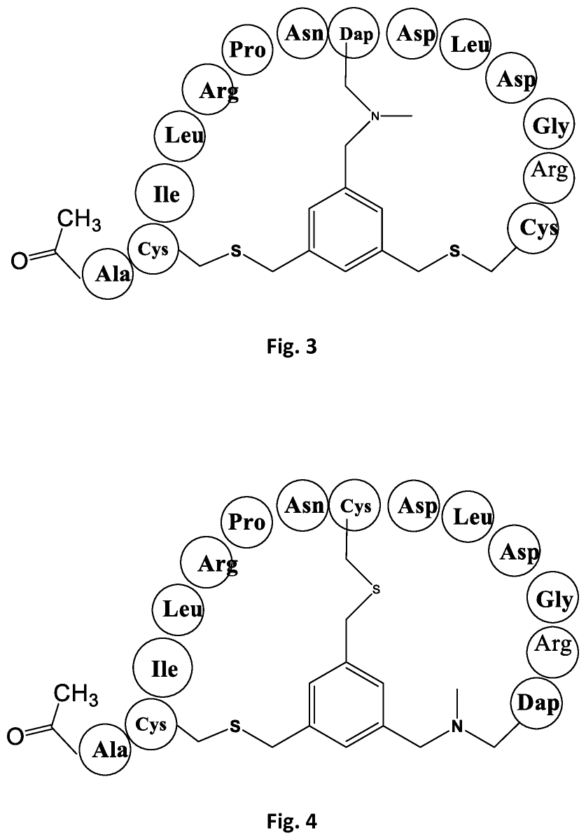

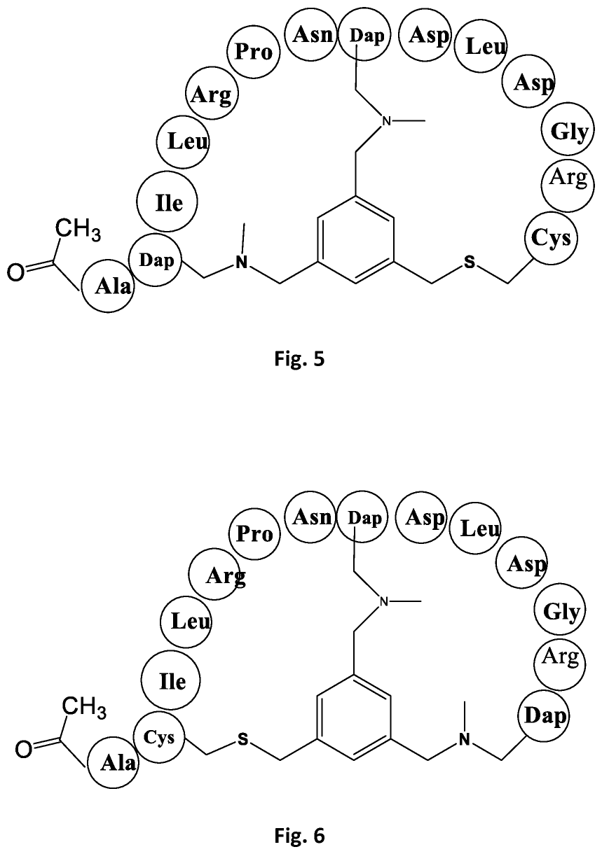

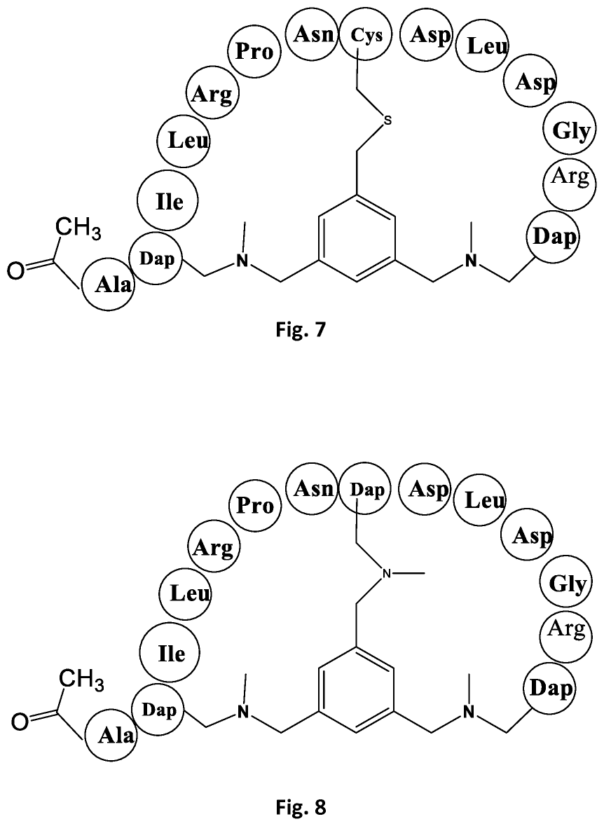

Bicycle peptide ligands according to the present invention were made corresponding to the bicycle region of the peptide ligand of Reference Example 1, with replacement of one, two or three cysteine residues by N-MeDAP residues forming alkylamino linkages to the TBMB scaffold. The structures of these derivative are shown schematically in FIGS. 2-8.

Cyclisation with TBMB was performed in a mixture of Acetonitrile/water in the presence of DIPEA as the base for 1-16 hours, as described in more detail in PCT/EP2017/083953 and PCT/EP2017/083954 filed 20 Dec. 2017. Unlike the cyclisation of Reference Example 1, the yield is relatively low when using the conventional NaHCO₃ as the base.

The measured Ki values are shown in Table 1. It can be seen that most of the examples exhibit high binding affinity to integrin αvβ3 which demonstrates that the change to alkylamino linkages in this example resulted in relatively little change in binding affinity relative to the thioether linked derivative of Reference Example 1. The sole exception is Example 8, in which replacement of all three thioether linkages by alkylamino linkages resulted in a larger drop in the measured affinity for integrin αvβ3.

TABLE 1
Integrin Binding Dap(Me) Substituted Bicycles

| Compound | | | Ki (nM) | Ki (nM) | Mean Ki |
|---|---|---|---|---|---|
| Identifier | Sequence | Peptide | n1 | n2 | (nM) |
| BCY00009174 | [Ac]ACILRPNCDLDGRCA | Ac-A-(43-06-00)(TBMB)-A | 10.0 | 10.8 | 10.4 |
| Reference | | | | | |
| Example 1 | | | | | |
| BCY00009175 | [Ac]A[Dap(Me)]ILRPNCDLDGRCA | Ac-A-(43-06-00)(TBMB)-A | 15.2 | 12.3 | 13.8 |
| Example 1 | | Cys1Dap(Me) | | | |
| BCY00009176 | [Ac]ACILRPN[Dap(Me)]DLDGRCA | Ac-A-(43-06-00)(TBMB)-A | 498 | 496 | 498 |
| Example 2 | | Cys2Dap(Me) | | | |
| BCY00009177 | [Ac]ACILRPNCDLDGR[Dap(Me)]A | Ac-A-(43-06-00)(TBMB)-A | 20.5 | 27.7 | 24.1 |
| Example 3 | | Cys3Dap(Me) | | | |
| BCY00009178 | [Ac]A[Dap(Me)]ILRPN[Dap(Me)] | Ac-A-(43-06-00)(TBMB)-A | 803 | 742 | 772 |
| Example 4 | DLDGRCA | Cys1Dap(Me) Cys2Dap(Me) | | | |
| BCY00009179 | [Ac]ACILRPN[Dap(Me)]DLDGR[Dap(Me)]A | Ac-A-(43-06-00)(TBMB)-A | 663 | 590 | 626 |
| Example 5 | | Cys2Dap(Me) Cys3Dap(Me) | | | |
| BCY00009180 | [Ac]A[Dap(Me)]ILRPNCDLDGR[Dap(Me)]A | Ac-A-(43-06-00)(TBMB)-A | 55.5 | 64.3 | 59.9 |
| Example 6 | | Cys1Dap(Me) Cys3Dap(Me) | | | |
| BCY00009181 | [Ac]A[Dap(Me)]ILRPN[Dap(Me)]DLDGR | Ac-A-(43-06-00)(TBMB)-A | >18.6 μM | >18.6 μM | >18.6 μM |
| Example 7 | [Dap(Me)]A | Cys1Dap(Me) Cys2Dap(Me) | | | |
| | | Cys3Dap(Me) | | | |

### Reference Examples A1-A31

The following reference peptide ligands having a TBMB or TATA scaffold with three thioether linkages to cysteine residues of the specified peptide sequences were prepared and evaluated for affinity to integrin αvβ3 as described in detail in our earlier application GB1800598.3 filed 15 Jan. 2018.

TABLE 2
Biological Assay Data for Reference Bicycle Ligands

| Reference | | | Molecular | |
|---|---|---|---|---|
| Example | Peptide | Structure | Scaffold | Ki (nM) |
| A1 | 43-01-00-N001 | ACLDHMECRGDMDCA | TBMB | 31.34 ± 8.63 |
| A2 | 43-04-00-N001 | ACYHAHRCDGGPFCA | TBMB | 59.7 n = 1 |
| A3 | 43-06-00-N001 | ACLHFSRCDGGMHCA | TBMB | 168 n = 1 |
| A4 | 43-06-00-N015 | Ac-CIL(HArg)PNCDLDGRCA-Sar10-(D-K) | TBMB | 1510 ± 235.2 |
| A5 | 43-06-00-N018 | Ac-CIL(HArg)PNCDLDGRCA-Sar6-(D-K) | TBMB | 14.8 ± 4.34 |
| A6 | 43-10-00-N001 | ACAGIVSCDGRPLCA | TBMB | 173 n = 1 |
| A7 | 43-16-00-N005 | ACKNFNPECLRGDSLCA | TBMB | 95.6 n = 1 |
| A8 | 43-16-00-N007 | ACHTRAHDCYWESIVCA | TBMB | 25.53 ± 14.83 |
| A9 | 43-23-00-N002 | ACKHYGRTDCHDTCA | TATA | 763 n = 1 |
| A10 | 43-24-00-N002 | ACDASRLDCVPSSSGCA | TATA | 121.15 ± 166.3 |
| A11 | 43-25-00-N002 | ACPHIGRTDCPPCA | TATA | 126.3 ± 93.49 |
| A12 | 43-26-00-N002 | ACYDDCRRLDHWQHSCA | TATA | 33.5 n = 1 |
| A13 | 43-27-00-N002 | ACRHSDRLDCLPCA | TATA | 10.7 n = 1 |
| A14 | 43-32-00-N002 | ACPHSLRLDCHDCA | TATA | 29.5 n = 1 |
| A15 | 43-33-00-N002 | ACELRHDLCRSHDHWCA | TATA | 81.9 n = 1 |
| A16 | 43-39-00-N002 | ACRHTHRLDCTESCA | TATA | 19.1 n = 1 |
| A17 | 43-40-00-N002 | ACPHAGRCDGPPTCA | TATA | 84.23 ± 26.4 |
| A18 | 43-40-00-N002 | ACPHAGRCDGPPTCA | TATA | 1090.33 ± 97.21 |
| A19 | 43-42-00-N002 | ACGHVGRLDCHIPCA | TATA | 864 ± 197.96 |
| A20 | 43-43-00-N002 | ACPHVHRLDCHAPCA | TATA | 727 ± 101.92 |
| A21 | 43-41-01-N001 | ACDHRRMDCPEVCA | TATA | 38 n = 1 |
| A22 | 43-41-02-N001 | ACDHRRMDCPTLCA | TATA | 42 n = 1 |
| A23 | 43-41-03-N001 | ACDHRRMDCPTNCA | TATA | 52 n = 1 |
| A24 | 43-41-04-N001 | ACDHTRMDCPHNCA | TATA | 107 n = 1 |
| A25 | 43-41-05-N001 | ACHVGRMDCFQECA | TATA | 563 n = 1 |
| A26 | 43-23-01-N001 | ACKHSGRTDCHDTCA | TATA | 613 n = 1 |
| A27 | 43-24-01-N001 | ACDASRLDCPYSVSLCA | TATA | 204 n = 1 |
| A28 | 43-24-02-N001 | ACDASRLDCPVVSHLCA | TATA | 93 n = 1 |
| A29 | 43-25-01-N001 | ACKHAGRTDCPPCA | TATA | 123 n = 1 |
| A30 | 43-25-02-N001 | ACRHAGRTDCPPCA | TATA | 153 n = 1 |
| A31 | 43-40-01-N001 | ACPHLGRCDGPLVCA | TATA | 682 n = 1 |

In view of the results obtained above in Examples 1-6, it is predicted that derivatives of the reference examples A1-A31 according to the present invention, i.e. having alkylamino linkages in place of one, two or three of the thioether linkages in the reference examples, will also display affinity for integrin αvβ3. All such derivatives having affinity for integrin αvβ3 are therefore included within the scope of the present invention.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A peptide ligand specific for integrin αvβ3 comprising a polypeptide comprising three residues selected from cysteine, L-2,3-diaminopropionic acid (Dap), N-beta-alkyl-L-2,3-diaminopropionic acid (N-AlkDap) and N-beta-haloalkyl-L-2,3-diaminopropionic acid (N-HAlkDap), with the proviso that at least one of said three residues is selected from Dap, N-AlkDap or N-HAlkDap, the said three residues being separated by at least two loop sequences, and a molecular scaffold, the peptide being linked to the scaffold by covalent alkylamino linkages with the Dap or N-AlkDap or N-HAlkDap residues of the polypeptide and by thioether linkages with the cysteine residues of the polypeptide when the said three residues include cysteine, such that two polypeptide loops are formed on the molecular scaffold.

2. The peptide ligand as defined in claim 1, wherein the peptide ligand comprises an amino acid sequence selected from:
Cᵢ-X₁-Cᵢᵢ-X₂-Cᵢᵢᵢ
wherein:
Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap; and
X₁ and X₂ represent the amino acid residues between the Cysteine, Dap, N-AlkDap or N-HAlkDap residues, wherein each of X₁ and X₂ independently has from 2 to 9 amino acid residues.

3. The peptide ligand as defined in any preceding claim, wherein two of Cᵢ, Cᵢᵢ, and Cᵢᵢᵢ are selected from Dap, N-AlkDap or N-HAlkDap, and the third one of Cᵢ, Cᵢᵢ, and Cᵢᵢᵢ is cysteine, preferably wherein Cᵢᵢ is cysteine.

4. The peptide ligand as defined in claim 1 or 2, wherein one of Cᵢ, Cᵢᵢ, and Cᵢᵢᵢ is selected from Dap, N-AlkDap or N-HAlkDap, and the others of Cᵢ, Cᵢᵢ, and Cᵢᵢᵢ are cysteine.

5. The peptide ligand as defined in any preceding claim, wherein the molecular scaffold is 1,3,5-tris(methylene)benzene (TBMB) or 1,1′,1″-(1,3,5-triazinane-1,3,5-triyl)triprop-2-en-1-one (TATA).

6. The peptide ligand as defined in claim 1 or claim 2, which comprises an amino acid sequence selected from:
(SEQ ID NO: 1)
CᵢLDHMECᵢᵢRGDMDCᵢᵢᵢ;
(SEQ ID NO: 2)
CᵢYHAHRCᵢᵢDGGPFCᵢᵢᵢ;
(SEQ ID NO: 3)
CᵢLHFSRCᵢᵢDGGMHCᵢᵢᵢ;
(SEQ ID NO: 4)
CᵢILRPNCᵢᵢDLDGRCᵢᵢᵢ;
(SEQ ID NO: 5)
CᵢIL(HArg)PNCᵢᵢDLDGRCᵢᵢᵢ;
(SEQ ID NO: 6)
CᵢAGIVSCᵢᵢDGRPLC_{iii;}
(SEQ ID NO: 7)
CᵢKNFNPECᵢᵢLRGDSLCᵢᵢᵢ;
(SEQ ID NO: 8)
CᵢHTRAHDCᵢᵢYWESIVCᵢᵢᵢ;
(SEQ ID NO: 12)
CᵢYDDCᵢᵢRRLDHWQHSC_{iii;}
(SEQ ID NO: 31)
Cᵢ-X-H-X-X-R-T/L-D-Cᵢᵢ-X-X-X₁-Cᵢᵢᵢ;
(SEQ ID NO: 32)
Cᵢ-D/E-A/L-S/R-R/H-L/D-D/L-Cᵢᵢ-X-X-X-X-S/H-X-Cᵢᵢᵢ;
(SEQ ID NO: 33)
Cᵢ-P-H-A/L-G-R-Cᵢᵢ-D-G-P-P/L-T/V-Cᵢᵢᵢ;
and
(SEQ ID NO: 34)
Cᵢ-D/H-H/V-X-R-M-D-Cᵢᵢ-P/F-X-X-Cᵢᵢᵢ;
wherein X represents any amino acid and X₁ represents any amino acid or is absent and Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap, or a pharmaceutically acceptable salt thereof.

7. The peptide ligand as defined in claim 6, wherein the peptide ligand of Cᵢ-X-H-X-X-R-T/L-D-Cᵢᵢ-X-X-X₁-Cᵢᵢᵢ (SEQ ID NO: 31) comprises an amino acid sequence selected from any one of SEQ ID NOS: 9, 11, 13-14, 16, 18-19, 25 and 28-29:
(SEQ ID NO: 9)
CᵢKHYGRTDCᵢᵢHDTCᵢᵢᵢ;
(SEQ ID NO: 11)
CᵢPHIGRTDCᵢᵢPPCᵢᵢᵢ;
(SEQ ID NO: 13)
CᵢRHSDRLDCᵢᵢLPCᵢᵢᵢ;
(SEQ ID NO: 14)
CᵢPHSLRLDCᵢᵢHDCᵢᵢᵢ;
(SEQ ID NO: 16)
CᵢRHTHRLDCᵢᵢTESCᵢᵢᵢ;
(SEQ ID NO: 18)
CᵢGHVGRLDCᵢᵢHIPCᵢᵢᵢ;
(SEQ ID NO: 19)
CᵢPHVHRLDCᵢᵢHAPCᵢᵢᵢ;
(SEQ ID NO: 25)
CᵢKHSGRTDCᵢᵢHDTCᵢᵢᵢ;
(SEQ ID NO: 28)
CᵢKHAGRTDCᵢᵢPPCᵢᵢᵢ;
and
(SEQ ID NO: 29)
CᵢRHAGRTDCᵢᵢPPCᵢᵢᵢ;
wherein Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap, or a pharmaceutically acceptable salt thereof.

8. The peptide ligand as defined in claim 6, wherein the peptide ligand of Cᵢ-D/E-A/L-S/R-R/H-L/D-D/L-Cᵢᵢ-X-X-X-X-S/H-X-Cᵢᵢᵢ (SEQ ID NO: 32) comprises an amino acid sequence selected from any one of SEQ ID NOS: 10, 15 and 26-27:
(SEQ ID NO: 10)
CᵢDASRLDCᵢᵢVPSSSGCᵢᵢᵢ;
(SEQ ID NO: 15)
CᵢELRHDLCᵢᵢRSHDHWCᵢᵢᵢ;
(SEQ ID NO: 26)
CᵢDASRLDCᵢᵢPYSVSLCᵢᵢᵢ;
and
(SEQ ID NO: 27)
CᵢDASRLDCᵢᵢPVVSHLCᵢᵢᵢ;
wherein Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap, or a pharmaceutically acceptable salt thereof.

9. The peptide ligand as defined in claim 6, wherein the peptide ligand of Cᵢ—P-H-A/L-G-R-Cᵢᵢ-D-G-P-P/L-T/V-Cᵢᵢ (SEQ ID NO: 33) comprises an amino acid sequence selected from any one of SEQ ID NOS: 17 and 30:
(SEQ ID NO: 17)
CᵢPHAGRCᵢᵢDGPPTCᵢᵢᵢ;
and
(SEQ ID NO: 30)
CᵢPHLGRCᵢᵢDGPLVCᵢᵢᵢ;
wherein Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap, or a pharmaceutically acceptable salt thereof.

10. The peptide ligand as defined in claim 6, wherein the peptide ligand of C-D/H-H/V-X-R-M-D-Cᵢᵢ—P/F-X-X-Cᵢᵢᵢ (SEQ ID NO: 34) comprises an amino acid sequence selected from any one of SEQ ID NOS: 20-24:
(SEQ ID NO: 20)
CᵢDHRRMDCᵢᵢPEVCᵢᵢᵢ;
(SEQ ID NO: 21)
CᵢDHRRMDCᵢᵢPTLCᵢᵢᵢ;
(SEQ ID NO: 22)
CᵢDHRRMDCᵢᵢPTNCᵢᵢᵢ;
(SEQ ID NO: 23)
CᵢDHTRMDCᵢᵢPHNCᵢᵢᵢ;
and
(SEQ ID NO: 24)
CᵢHVGRMDCᵢᵢFQECᵢᵢᵢ;
wherein Cᵢ, Cᵢᵢ, and Cᵢᵢᵢ are independently cysteine, L-2,3-diaminopropionic acid (Dap), N-beta-alkyl-L-2,3-diaminopropionic acid (N-AlkDap), or N-beta-haloalkyl-L-2,3-diaminopropionic acid (N-HAlkDap), provided that at least one of Cᵢ, Cᵢᵢ, and Cᵢᵢᵢ is Dap, N-AlkDap or N-HAlkDap, or a pharmaceutically acceptable salt thereof.

11. The peptide ligand as defined in any one of claims 1, 2 or 6, which comprises an amino acid sequence selected from:
A-(SEQ ID NO: 1)-A (herein referred to as 43-01-00-N001);
A-(SEQ ID NO: 2)-A (herein referred to as 43-04-00-N001);
A-(SEQ ID NO: 3)-A (herein referred to as 43-06-00-N001);
A-(SEQ ID NO: 4)-A (herein referred to as 43-06-00-N017);
Ac-(SEQ ID NO: 5)-A-Sar₁₀-D-K (herein referred to as 43-06-00-N015);
Ac-(SEQ ID NO: 5)-A-Sar₆-D-K (herein referred to as 43-06-00-N018);
A-(SEQ ID NO: 6)-A (herein referred to as 43-10-00-N001);
A-(SEQ ID NO: 7)-A (herein referred to as 43-16-00-N005);
A-(SEQ ID NO: 8)-A (herein referred to as 43-16-00-N007);
A-(SEQ ID NO: 9)-A (herein referred to as 43-23-00-N002);
A-(SEQ ID NO: 10)-A (herein referred to as 43-24-00-N002);
A-(SEQ ID NO: 11)-A (herein referred to as 43-25-00-N002);
A-(SEQ ID NO: 12) (herein referred to as 43-26-00-N002);
A-(SEQ ID NO: 13) (herein referred to as 43-27-00-N002);
A-(SEQ ID NO: 14)-A (herein referred to as 43-32-00-N002);
A-(SEQ ID NO: 15)-A (herein referred to as 43-33-00-N002);
A-(SEQ ID NO: 16)-A (herein referred to as 43-39-00-N002);
A-(SEQ ID NO: 17)-A (herein referred to as 43-40-00-N002);
A-(SEQ ID NO: 18)-A (herein referred to as 43-42-00-N002);
A-(SEQ ID NO: 19)-A (herein referred to as 43-43-00-N002);
A-(SEQ ID NO: 20)-A (herein referred to as 43-41-01-N001);
A-(SEQ ID NO: 21)-A (herein referred to as 43-41-02-N001);
A-(SEQ ID NO: 22)-A (herein referred to as 43-41-03-N001);
A-(SEQ ID NO: 23)-A (herein referred to as 43-41-04-N001);
A-(SEQ ID NO: 24)-A (herein referred to as 43-41-05-N001);
A-(SEQ ID NO: 25)-A (herein referred to as 43-23-01-N001);
A-(SEQ ID NO: 26)-A (herein referred to as 43-24-01-N001);
A-(SEQ ID NO: 27)-A (herein referred to as 43-24-02-N001);
A-(SEQ ID NO: 28)-A (herein referred to as 43-25-01-N001);
A-(SEQ ID NO: 29)-A (herein referred to as 43-25-02-N001); and
A-(SEQ ID NO: 30)-A (herein referred to as 43-40-01-N001).

12. The peptide ligand as defined in claim 11, wherein the molecular scaffold is selected from 1,3,5-tris(bromomethyl)benzene (TBMB) and the peptide ligand comprises an amino acid sequence selected from:
A-(SEQ ID NO: 1)-A (herein referred to as 43-01-00-N001);
A-(SEQ ID NO: 2)-A (herein referred to as 43-04-00-N001);
A-(SEQ ID NO: 3)-A (herein referred to as 43-06-00-N001);
A-(SEQ ID NO: 4)-A (herein referred to as 43-06-00-N017);
Ac-(SEQ ID NO: 5)-A-Sar₁₀-D-K (herein referred to as 43-06-00-N015);
Ac-(SEQ ID NO: 5)-A-Sar₆-D-K (herein referred to as 43-06-00-N018);
A-(SEQ ID NO: 6)-A (herein referred to as 43-10-00-N001);
A-(SEQ ID NO: 7)-A (herein referred to as 43-16-00-N005); and
A-(SEQ ID NO: 8)-A (herein referred to as 43-16-00-N007).

13. The peptide ligand as defined in claim 11 or claim 12, wherein the molecular scaffold is selected from 1,3,5-tris(bromomethyl)benzene (TBMB) and the peptide ligand comprises an amino acid sequence selected from:
Ac-(SEQ ID NO: 5)-A-Sar₆-D-K (herein referred to as 43-06-00-N018).

14. The peptide ligand as defined in claim 11, wherein the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 9)-A (herein referred to as 43-23-00-N002);
A-(SEQ ID NO: 10)-A (herein referred to as 43-24-00-N002);
A-(SEQ ID NO: 11)-A (herein referred to as 43-25-00-N002);
A-(SEQ ID NO: 12) (herein referred to as 43-26-00-N002);
A-(SEQ ID NO: 13) (herein referred to as 43-27-00-N002);
A-(SEQ ID NO: 14)-A (herein referred to as 43-32-00-N002);
A-(SEQ ID NO: 15)-A (herein referred to as 43-33-00-N002);
A-(SEQ ID NO: 16)-A (herein referred to as 43-39-00-N002);
A-(SEQ ID NO: 17)-A (herein referred to as 43-40-00-N002);
A-(SEQ ID NO: 18)-A (herein referred to as 43-42-00-N002);
A-(SEQ ID NO: 19)-A (herein referred to as 43-43-00-N002);
A-(SEQ ID NO: 20)-A (herein referred to as 43-41-01-N001);
A-(SEQ ID NO: 21)-A (herein referred to as 43-41-02-N001);
A-(SEQ ID NO: 22)-A (herein referred to as 43-41-03-N001);
A-(SEQ ID NO: 23)-A (herein referred to as 43-41-04-N001);
A-(SEQ ID NO: 24)-A (herein referred to as 43-41-05-N001);
A-(SEQ ID NO: 25)-A (herein referred to as 43-23-01-N001);
A-(SEQ ID NO: 26)-A (herein referred to as 43-24-01-N001);
A-(SEQ ID NO: 27)-A (herein referred to as 43-24-02-N001);
A-(SEQ ID NO: 28)-A (herein referred to as 43-25-01-N001);
A-(SEQ ID NO: 29)-A (herein referred to as 43-25-02-N001); and
A-(SEQ ID NO: 30)-A (herein referred to as 43-40-01-N001).

15. The peptide ligand as defined in any of claims 1 to 5, wherein the peptide ligand comprises an amino acid sequence selected from one or more of the peptide ligand sequences A1-A31 listed in Table 2, or a pharmaceutically acceptable salt thereof, with the proviso that one or more of the cysteine residues in said peptide ligand sequences A1-A31 is replaced by Dap, N-AlkDap or N-HAlkDap.

16. The peptide ligand as defined in any one of claims 1 to 15, wherein the integrin αvβ3 is human integrin αvβ3.

17. A drug conjugate comprising a peptide ligand as defined in any one of claims 1 to 15, conjugated to one or more effector and/or functional groups.

18. The drug conjugate comprising a peptide ligand as defined in any one of claims 1 to 15, conjugated to one or more cytotoxic agents.

19. The drug conjugate as defined in claim 18, wherein said cytotoxic agent is selected from DM-1 and MMAE.

20. A pharmaceutical composition which comprises the peptide ligand of any one of claims 1 to 15 or the drug conjugate of any one of claims 16 to 19, in combination with one or more pharmaceutically acceptable excipients.

21. The peptide ligand as defined in any one of claims 1 to 15 or the drug conjugate as defined in any one of claims 16 to 19, for use in preventing, suppressing or treating a disease or disorder mediated by integrin αvβ3.
